(19) United States
(12) Patent Application Publication
(10) Pub. No.: US 2011/0270029 A1
(43) Pub. Date: Nov. 3, 2011

(54) GASTRIC BAND WITH ELECTRICAL STIMULATION

(76) Inventors: Janel A. Birk, Oxnard, CA (US); Sean Snow, Carpinteria, CA (US)

(21) Appl. No.: 13/184,387

(22) Filed: Jul. 15, 2011

Related U.S. Application Data
(63) Continuation of application No. 12/853,498, filed on Aug. 10, 2010.
(60) Provisional application No. 61/237,881, filed on Aug. 28, 2009.

(51) Int. Cl. A61F 2/04; A61N 1/36

(52) U.S. Cl. 600/37; 607/3

(57) ABSTRACT

A gastric band system including a functional electrical stimulation component is provided. Stimulation electrodes on the gastric band may be used to stimulate the vagal nerve and/or splanchnic nerve, which can inhibit the patient's appetite. The gastric band may have an inflatable member for adjusting a stoma size. The stimulation electrodes may be mounted on the inflatable member. The system may include a controller including a pressure sensor for monitoring the hydraulic pressure within the inflatable inner member and for controlling the stimulation component.

### CROSS REFERENCE TO RELATED APPLICATIONS

This application is a continuation application of U.S. patent application Ser. No. 12/853,498, filed on Aug. 10, 2010, which claims the benefit and priority of U.S. Provisional Application No. 61/237,881, filed on Aug. 28, 2009. The entire contents of each of these applications are hereby incorporated by reference herein.

### FIELD OF THE INVENTION

The present invention relates, in general, to devices and methods for controlling obesity, and, more particularly, to a gastric band or gastric band assembly/system that provides ongoing adjustment of stoma size in a patient in conjunction with electrical stimulation of the stomach.

### BACKGROUND OF THE INVENTION

Severe obesity is an increasingly prevalent chronic condition that is difficult for physicians to treat in their patients through diet and exercise alone. Gastrointestinal surgery is used by physicians to treat people who are severely obese and cannot lose weight by traditional means or who suffer from serious obesity-related health problems. Generally, gastrointestinal surgery promotes weight loss by restricting food intake, and more specifically, restrictive operations limit food intake by creating a narrow passage or “stoma” from the upper part of the stomach into the larger lower part, which reduces the amount of food the stomach can hold and slows the passage of food through the stomach. Initially, the stoma was of a fixed size, but physicians have more recently determined that the procedure is more effective if the stoma can be adjusted to alter its size.

One of the more commonly used of these purely restrictive operations for obesity is adjustable gastric banding (AGB). In an exemplary AGB procedure, a hollow band (i.e., a gastric band) made of silicone elastomer is placed around the stomach near its upper end, creating a small pouch and a narrow passage (i.e., a stoma) into the rest of the stomach. The band is then inflated with a saline solution by using a non-coring needle and syringe to access a small port that is placed under the skin. To control the size of the stoma, the gastric band can be tightened or loosened over time by the physician or another technician extracorporeally by increasing or decreasing the amount of saline solution in the band via the access port to change the size of the passage or stoma.

Providing fine adjustments of the gastric band after initial stoma sizing has proven a significant improvement in the adjustable gastric banding procedure. However, there is an ongoing difficulty in determining when to further adjust the gastric band and how much to increase or decrease the band's size or diameter to achieve a desired stoma size. Numerous gastric bands have been developed to allow a physician or other technician to adjust an implanted gastric band. In general, these band systems include a sensor for measuring or determining parameters associated with the patient and in response, the physician or technician acts to adjust the volume of fluid in the band based on the patient parameters. For example, one adjustable gastric band system determines when the pressure in a patient's stomach exceeds a pre-set limit and provides an alarm to an external control device. A doctor or other operator then responds by loosening the gastric band by removing an amount of fluid from the band via the external access port and fill line.

In another gastric band system, disclosed in U.S. Patent Application No. 20060089571 to Gertner, gastric bands may operate in conjunction with electrical stimulation of the stomach. In one embodiment, a transgastric fastening assembly serves to reduce the volume of the stomach as well as provide for electrical stimulation. An electrical signal runs through electrodes in the transgastric fastener assembly to possibly alter the contraction patterns of the stomach or to electrically create a feeling of satiety in addition to reducing the volume of the stomach and creating a restriction to flow in the stomach.

Due to certain limitations of existing technologies, there remains a need for an improved gastric banding system, and associated adjustment methods, for providing improved adjustments to the size of a stoma in a patient being treated for obesity.

### BRIEF DESCRIPTION OF THE DRAWINGS

Features and advantages of the present invention will become appreciated as the same become better understood with reference to the specification, claims, and appended drawings wherein:

FIG. 1 are schematic views of two embodiments of a gastric band system that provides ongoing adjustment of stoma size in a patient in conjunction with electrical stimulation of the stomach; and

FIG. 2 are a number of views of an exemplary gastric band having an uneven inner circumference on which are mounted a series of stimulation electrodes.

### DETAILED DESCRIPTION OF THE PREFERRED EMBODIMENTS

The present application provides a gastric band or band system that incorporates a functional electrical stimulation system. The resulting implantable medical device provides the treatment of a gastric band with the treatment of functional electrical stimulation of the nervous system.

A gastric band includes any number of devices in contact with the upper surfaces of the stomach, such as the cardia region. The stomach surface must be sufficiently broad to provide adequate surface area for holding stimulation electrodes. When current passes through the electrodes the vagal nerve and/or splanchnic nerve are stimulated, which can inhibit the patient's appetite.

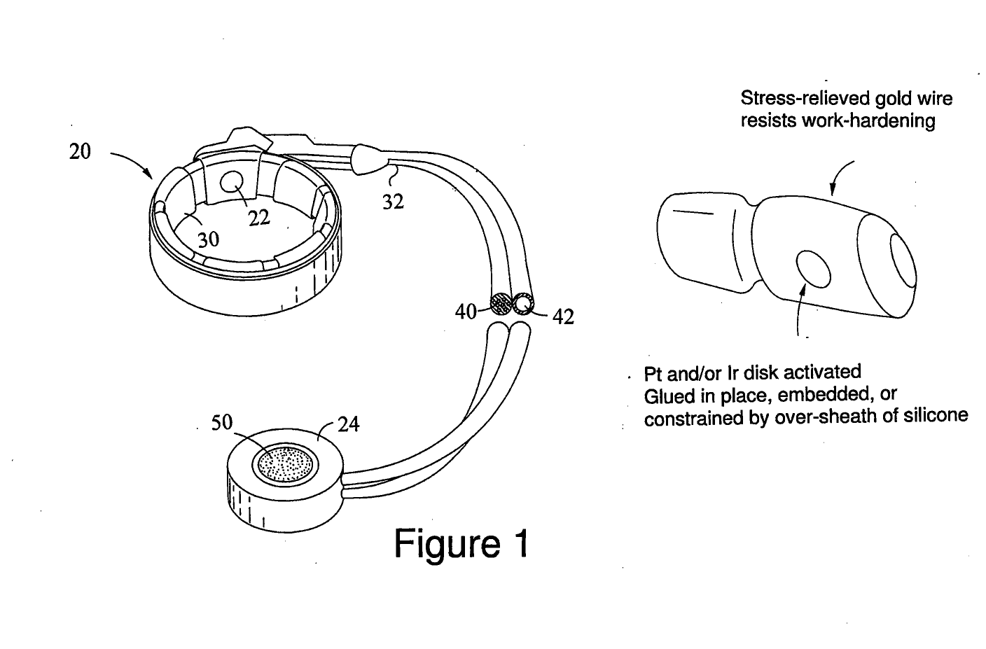

As seen in FIG. 1, a gastric band functional electrical stimulation system includes a gastric band 20 on which is placed one or more electrodes 22, and a controller 24. The gastric band 20 includes a generally circular band member 30 to which a flexible cord 32 tangentially attaches. The single or multiple stimulation electrodes 22 are positioned on an inner surface of the band member 30. When implanted, the band member 30 surrounds and contacts an upper region of the stomach, such as the stoma or cardia. The inner surface may be relatively solid, or may be an inflatable ring for adjusting the amount of constriction. Several prominent nerves extend through the upper portions of the stomach, and stimulating them with current from the electrodes may induce feelings of satiety (fullness) or nausea, either of which inhibits the appetite.

A number of different gastric bands are available today, and the present invention may be used with any number of these as well as others not yet marketed. For example, a preferred gastric band for use is sold under the name LAP-BAND.RTM. Adjustable Gastric Banding System by Allergan, Inc. of Irvine, Calif., and is designed to be placed laparoscopically (via small incisions in the abdomen, usually 0.5-1.5 centimeters in length). An inflatable band is placed around the top portion of the patient's stomach, creating a small pouch that limits or reduces food consumption. The LAP-BAND.RTM. System is adjustable, which means that the inflatable band can be tightened or loosened to help the patient achieve a level of satiety while maintaining a healthy diet, supporting a patient's long-term weight loss success. Other possible gastric bands are adjustable electromechanically without hydraulics, and still others may have a fixed-size with no adjustment.

The electrodes 22 may serve as the source or current return, i.e., as the anode or cathode. Each electrode receives current from or transmits current to the controller 24 via insulated leads or wires 40. The wires 40 pass along the flexible cord 32, in parallel to a tubular conduit 42 having a lumen for flow of fluid, typically saline. Fluid may be added or removed from within a balloon on the interior of the gastric band 20 to adjust constriction of the stomach. The wires 40 may be encompassed by a polymer jacket that is adhered to or molded with the tubular conduit 42. The wires 40 are formed into a geometry which provides strain relief and resists fracturing.

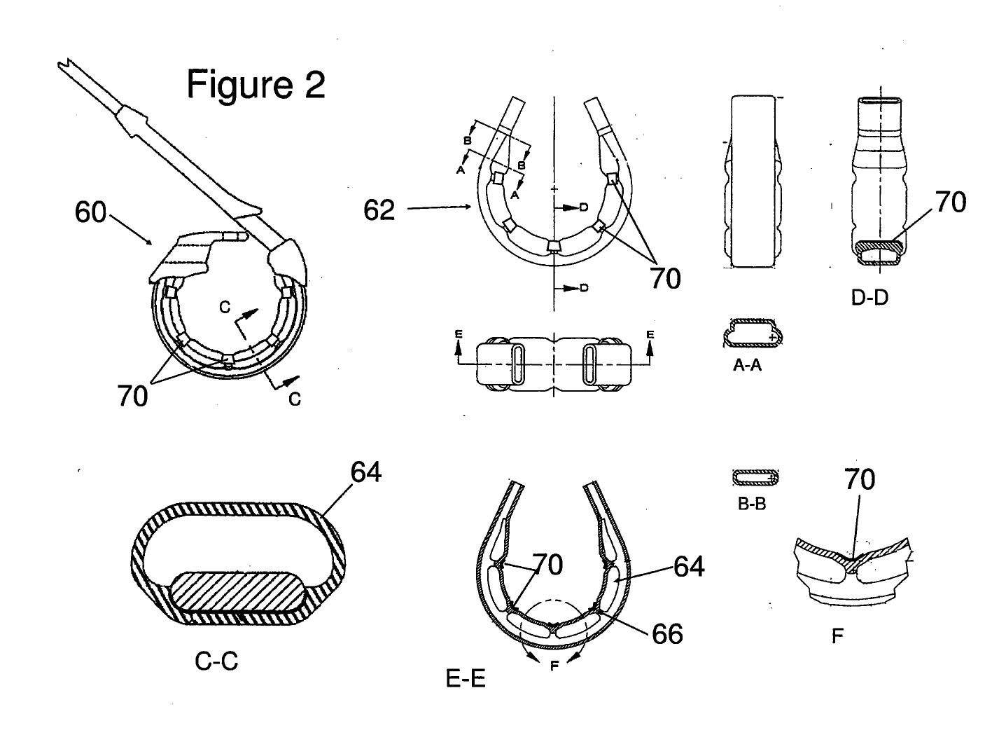

The electrodes 22 are preferably formed of thin plates of suitably conductive and biocompatible material, such as platinum (Pt), Iridium (Ir), platinum/iridium alloy, tantalum, etc. The electrodes 22 may be arranged linearly on the surface of the gastric band member 30 or in a two-dimensional pattern. If the gastric band has a non-uniform inner circumference, the electrodes 22 may be arranged in a three-dimensional pattern, as shown in FIG. 2 and described below.

The controller 24 may include an access port 50 for adjusting the gastric band 20 percutaneously. The controller 24 incorporates a power source and circuitry form controlling and delivering precise pulses of electrical current to the electrodes 22 on the inner face of the gastric band member 30. In one embodiment, the external case of the controller 24 is conductive and functions as a return electrode for the gastric band electrodes 22.

In a preferred embodiment, the system further includes one or more sensors to obtain physiological information and in turn operate the gastric band 20 and electrodes 22. For example, one or more pressure sensors may be provided for monitoring the hydraulic pressure within the gastric band balloon. The hydraulic pressure may be measured within the lumen of the tubular conduit 42 or a sensor may be incorporated into the band 20 itself, with a wire or wireless interface to the controller 24. The pressure information can be used diagnostically by the physician or can be used to control the electrical stimulation.

In one mode of operation, the gastric band functions normally with adjustments made to the level of constriction through fluid transfer, either by percutaneous addition through the access port 50 or by a needle-free telemetric system that utilizes an implantable fluid pump(s) and reservoir(s).

The functional electrical stimulation system operates by applying a precisely-controlled voltage across the source and return electrode. The two electrodes may both be on the inner surface of the band member 30, or one may be remote, such as on the controller 24. The potential difference across the electrodes creates a current flow through the tissue in contact with the electrodes of a desired duration and amplitude. The type of electrodes and signals used varies depending on desired effect. Choices include: monopolar or bipolar delivery, monophasic and biphasic charge pulses, interphase intervals, active and passive charge recovery, variable and fixed frequency, symmetric and asymmetric phases, and various waveform shapes. As current flows through the tissue, the neurons located therein experience depolarization and, ultimately, activation. The action potentials are then conducted by the neurons to the regions of the body which influence feelings of satiety or nausea.

The functional electrical stimulation pulses may be programmed to follow a number of different protocols. For instance, the pulses may be activated on a timing system, such as on a daily schedule at documented times when the patient experiences hunger cravings. Alternatively, the pulses may be controlled on the basis of feedback from the band pressure monitoring system. For instance, pressure variations within the gastric band 20 may indicate the ingestion of food, which acts to raise the pressure within the gastric band balloon. The controller 24 may be programmed to detect such pressure changes and fire the stimulation pulses to thereby reduce the patient's appetite at the time of eating.

FIG. 2 illustrates one embodiment of a gastric band 60 that has an inner balloon 62 having a non-uniform periphery. Specifically, the balloon 62 features a series of contiguous chambers defined by alternating pillows 64 and depressions 66, as seen in Figure E-E. A series of stimulation electrodes 70, five in the illustrated embodiment, are positioned in the troughs at the depressions 66. In this way, the stimulation electrodes 70 are recessed and do not present a source of irritation or discomfort to the patient.

Although the invention has been described and illustrated with a certain degree of particularity, it is understood that the present disclosure has been made only by way of example, and that numerous changes in the combination and arrangement of parts can be resorted to by those skilled in the art without departing from the scope of the invention, as hereinafter claimed.

What is claimed is:

1. A gastric band electrical stimulation system for implantation in a body of an overweight patient, the stimulation system comprising:
a gastric band having an inner surface for placement around an upper portion of a stomach of the patient;
a stimulation electrode coupled to the inner surface of the gastric band and configured to provide electrical stimulation to the stomach of the patient for inhibiting appetite; and
a controller connected to the stimulation electrode for controlling the electrical stimulation to the stomach of the patient based upon a predetermined time when the patient is expected to experience hunger.

2. The stimulation system of claim 1 further comprising a second electrode coupled to the stimulation electrode and wherein the controller is configured to control a voltage between the stimulation electrode and the second electrode for flowing a current between the stimulation electrode and the second electrode.

3. The stimulation system of claim 2 wherein the second electrode is coupled to the inner surface of the gastric band.

4. The stimulation system of claim 2 wherein the second electrode is positioned remotely from the gastric band.

5. The stimulation system of claim 4 wherein the second electrode comprises a conductive case for enclosing the controller.

6. The stimulation system of claim 1 wherein the inner surface of the gastric band comprises an inflatable ring configured to adjust an amount of constriction on the upper portion of the stomach of the patient.

7. The stimulation system of claim 6 further comprising:
a conduit coupled to the gastric band and in fluid communication with the inflatable ring; and
a wire connected to the stimulation electrode and encompassed by an insulator, the insulator adhered with the conduit.

8. The stimulation system of claim 7 wherein the insulator is a polymer.

9. The stimulation system of claim 7 further comprising a plurality of stimulation electrodes coupled to the inflatable ring of the gastric band and a plurality of wires, each of the plurality of stimulation electrodes connected to one of the plurality of wires, the plurality of wires geometrically configured within an insulator to provide strain relief or resist fracturing, the insulator adhered with the conduit.

10. The stimulation system of claim 1 wherein the stimulation electrode is formed of a material selected from a group consisting of platinum, iridium, platinum/iridium alloy, and tantalum.

11. The stimulation system of claim 1 wherein the electrical stimulation to the stomach is a type of signal selected from a group consisting of monopolar or bipolar delivery, monophasic or biphasic charge pulses, interphase intervals, active and passive charge recovery, variable or fixed frequency, symmetric or asymmetric phases, and predetermined waveform shapes.

12. The stimulation system of claim 1 wherein the inner surface of the gastric band forms a recess thereon, the stimulation electrode positioned in the recess.

13. A gastric band electrical stimulation system for implantation in a patient for the treatment of obesity, the stimulation system comprising:
a gastric band configured to encircle an upper portion of a stomach of the patient;
an electrode coupled to the gastric band and configured to contact the upper portion of the stomach of the patient to electrically stimulate the stomach for inhibiting appetite;
a controller connected to the electrode and configured to control the electrical stimulation to the stomach based upon physiological information related to the patient; and
a sensor interfacing with the controller, the sensor configured to obtain the physiological information related to the patient.

14. The stimulation system of claim 13 wherein the physiological information comprises a pressure variation within the gastric band.

15. The stimulation system of claim 14 wherein the sensor is incorporated into the gastric band.

16. The stimulation system of claim 15 wherein the sensor wirelessly interfaces with the controller.

17. The stimulation system of claim 13 wherein the gastric band has an inflatable member, the inflatable member configured to contact the stomach of the patient when the gastric band is encircling the upper portion of the stomach of the patient.

18. The stimulation system of claim 17 wherein the physiological information comprises a hydraulic pressure within the inflatable member of the gastric band.

19. The stimulation system of claim 17 further comprising:
a conduit coupled with the gastric band for flowing a fluid; and
a wire connected to the electrode and surrounded by an insulator, the insulator disposed in parallel with the conduit.

20. The stimulation system of claim 17 wherein the inflatable member has a non-uniform inner circumference including a depression, the electrode positioned in the depression.
